(19) United States
(12) Patent Application Publication
(10) Pub. No.: US 2013/0289724 A1
(43) Pub. Date: Oct. 31, 2013

(54) AMNION AND CHORION WOUND DRESSING AND USES THEREOF

(71) Applicant: AFCELL MEDICAL, Parsippany, NJ (US)

(72) Inventor: Robin R. YOUNG, Wayne, PA (US)

(73) Assignee: AFCELL MEDICAL, Parsippany, NJ (US)

(21) Appl. No.: 13/804,731

(22) Filed: Mar. 14, 2013

Related U.S. Application Data
(60) Provisional application No. 61/638,255, filed on Apr. 25, 2012.

(51) Int. Cl. A61F 2/10

(52) U.S. Cl. CPC A61F 2/105
USPC 623/15.12; 156/281

(57) ABSTRACT

An improved wound dressing is described. The wound dressing contains an allograft having at least one layer of human amnion and chorion tissues that has a size and shape appropriate for covering a wound, a base sheet for securing the allograft over the wound, and optionally a support layer to protect the allograft. Methods of preparing the wound dressing and using it in wound healing are also described. The products and methods improve the performance of the wound healing, e.g., by accelerating wound healing, reducing scar formation, inflammation and risk of infection.

### CROSS-REFERENCE TO RELATED APPLICATIONS

This application is entitled to priority pursuant to 35 U.S.C. §119(e) to U.S. Provisional Patent Application No. 61/638,255, filed Apr. 25, 2012, which is hereby incorporated by reference herein in its entirety.

### BACKGROUND OF THE INVENTION

1. Field of Invention

Embodiments of the present invention relate to methods and products for improving wound healing. In particular, embodiments of the present invention relate to a wound dressing comprising an allograft having at least one layer of amnion and chorion tissues. The wound dressing are adapted for the ease of application to a wound to promote wound healing and prevent infection.

2. Background of the Invention

Wound dressings have been used to promote wound healing and prevent infection. For example, wound dressing can be used to stem bleeding, absorb exudate, ease pain, protect from infection, promote healing and may also reduce scar formation. Various materials have been used in wound dressings, such as cloth, leaves, honey, gauzes, films, gels, foams, polysaccharide pastes, granules and beads, etc. Wound dressings can be impregnated with an agent, such as an antiseptic agent, for further protection from bacteria or viral infection and improved wound healing. They may also be treated to prevent the wound from adhering to the dressings.

The amnion is a thin, cellular, extraembryonic collagen matrix that forms the inner membrane of a closed placental sac surrounding and protecting an embryo in reptiles, birds, and mammals. The sac contains the fetus and amniotic fluid or liquor amnii, in which the embryo is immersed, nourished and protected. Amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid.

The amnion has multiple functions, e.g., as a covering epithelium, as an active secretary epithelium, and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford et al., 1979, Am J Obstet Gynecol, 134:833-845. Amnioplastin, an isolated and chemically processed amniotic membrane, was used for continual dural repair, peripheral nerve injuries, conjunctival graft and flexor and muscle repair. See e.g., Chao et al., 1940, The British Medical Journal, Mar. 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In recent years, there have been renewed interests in the application of amnion in ocular surface reconstruction, for example, as an allograph for repairing corneal defects. See, for example, Tsai and Tseng, Cornea. 1994 September; 13(5):389-400; and Dua et al., Br. J. Ophthalmol 1999, 83:748-20 752. In addition, amnion and amniotic fluid have recently been used as sources of placental stem cells. See, e.g., U.S. Pat. No. 7,255,879 and WO 200073421.

Despite the clinical and published record regarding the safety and efficacy of amnion in broad surgical use, issues regarding reproducibility, safety and the precise form of amnion for each prospective indication have prevented amnion from achieving broad commercial distribution.

There is a need of improved methods and products that would enhance wound healing, effectively reduce inflammation and inhibit fibroblast formation, scarring and adhesion formation. The present invention relates to such improved methods and products.

It is now discovered that using amnion and chorine tissues in wound dressings as described in the present invention significantly enhances wound healing, reduces scar formation, inflammation and risk of infection.

### BRIEF SUMMARY OF THE INVENTION

In one general aspect, the present invention relates to a wound dressing, comprising:

(1) an allograft having at least one layer of human amnion and chorion tissues and a size and shape appropriate for placing inside boundaries of a wound; and

(2) a base sheet having an adhesive on a first major surface of the base sheet and a central opening extending therethrough, wherein the opening has a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound; and

(3) optionally a support layer mounted on a second major surface of the base sheet opposite to the first major surface.

In another general aspect, the present invention relates to a method of preparing a wound dressing. The method comprises:

drying an allograft comprising at least one layer of human amnion and chorion tissues over a frame having a shape appropriate for placing inside boundaries of a wound;

obtaining a base sheet having an adhesive on a first major surface of the base sheet and a central opening extending therethrough, the opening having a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound;

sterilizing the allograft and the base sheet; and

assembling the allograft and the base sheet in a package.

In one embodiment of the present invention, the method further comprises obtaining a support layer and assembling the support layer together with the allograft and the base sheet.

Another general aspect of the present invention relates to a method of improving wound healing, comprising applying to a wound a wound dressing comprising:

(1) an allograft having at least one layer of human amnion and chorion tissues and a size and shape appropriate for placing inside boundaries of a wound; and

(2) a base sheet having an adhesive on a first major surface of the base sheet and a central opening extending therethrough, wherein the opening has a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound; and

(3) optionally a support layer mounted on a second major surface of the base sheet opposite to the first major surface.

Yet another general aspect of the present invention relates to a kit, the kit comprising a plurality of wound dressings according to the present invention and instructions on using the wound dressings in wound healing, wherein at least two of the wound dressings are different in at least one of size and shape.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates a round shaped wound dressing according to an embodiment of the present invention, A: an allograft; B: a base sheet; C: the wound dressing containing the allograft and the base sheet (top view); and (D) the wound dressing containing a support layer, the allograft, the base sheet (side view);

FIG. 2 illustrates a rectangular shaped wound dressing according to an embodiment of the present invention, A: an allograft; B: a base sheet; and C: the wound dressing containing the allograft and the base sheet (top view);

FIG. 3 illustrates a triangular shaped wound dressing according to an embodiment of the present invention, A: an allograft; B: a base sheet; and C: the wound dressing containing the allograft and the base sheet (top view); and

FIG. 4 illustrates other wound dressings according to embodiments of the present invention, the wound dressings contain an allograft and a base sheet (top view).

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

The term “wound dressing” as used herein refers to a material used for covering or protecting a wound. For example, a wound dressing can be a surgical dressing, a bandage, or any other material suitable for covering or protecting a wound.

A “wound” as used herein can be any burn, cut, sore, blister, rash, or any other lesion or injury to skin or another external surface of a subject.

As used herein, the terms “boundary” and “boundaries” refer to the perimeter of a wound that defines the barrier between the wound and the skin surrounding the wound.

In one general aspect, the present invention relates to a wound dressing for improved wound healing. The wound dressing provides therapeutic angiogenic properties to the wound which stimulate capillary growth to accelerate healing, anti-microbial and anti-inflammatory functions to prevent infections and relieve pain during wound healing.

A wound dressing according to an embodiment of the present invention comprises: (1) an allograft having at least one layer of human amnion and chorion tissues and a size and shape appropriate for placing within boundaries of a wound; and (2) a base sheet having an adhesive on a first major surface of the base sheet and a central opening extending therethrough, wherein the opening has a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound. Optionally, the wound dressing can further comprise a support layer mounted on a second major surface of the base sheet opposite to the first major surface.

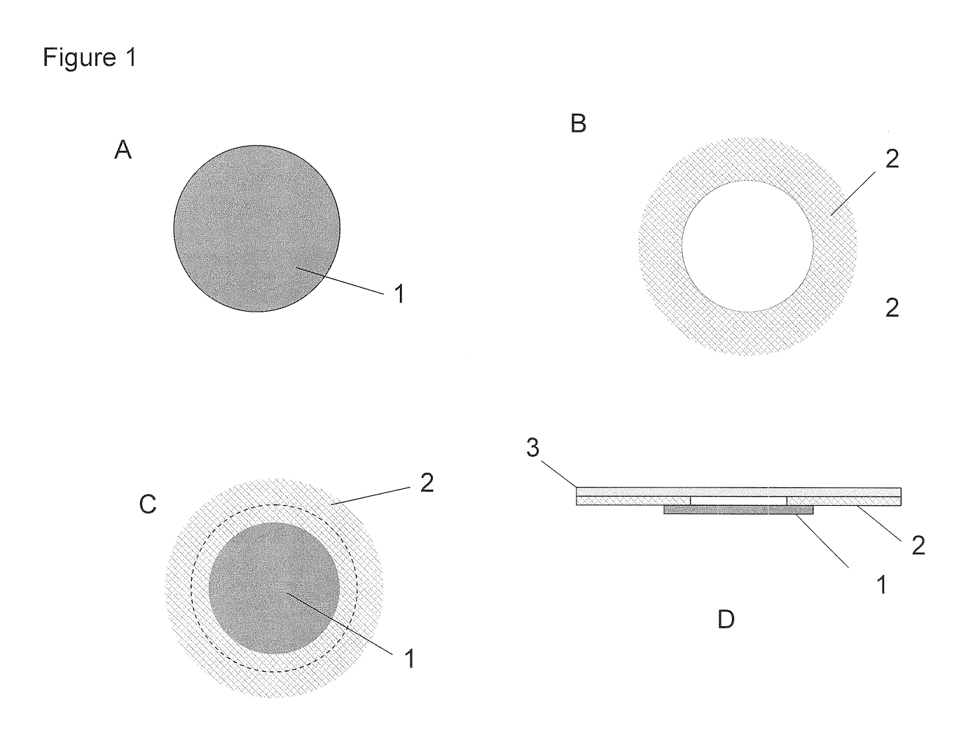

FIG. 1 illustrates a wound dressing according to an embodiment of the present invention. FIG. 1A illustrates a round or dot shaped allograft (1) having at least one layer of human amnion and chorion tissues. FIG. 1B illustrates a donut shaped base sheet (2) having an adhesive on one of its major surfaces. FIG. 1C illustrates a top view of a wound dressing containing the allograft (1) and the base sheet (2). FIG. 1D illustrates a side view of a wound dressing containing the allograft (1), the base sheet (2), and a support layer (3). The diameter of the wound dressing can be, e.g., 0.5 cm to 2 cm.

The wound dressing according to FIG. 1 can be useful for wound healing of various lesions. For example, it can be used for herpes lesions on either genital or oral areas. The wound dressing can adhere to wet, mucous surfaces.

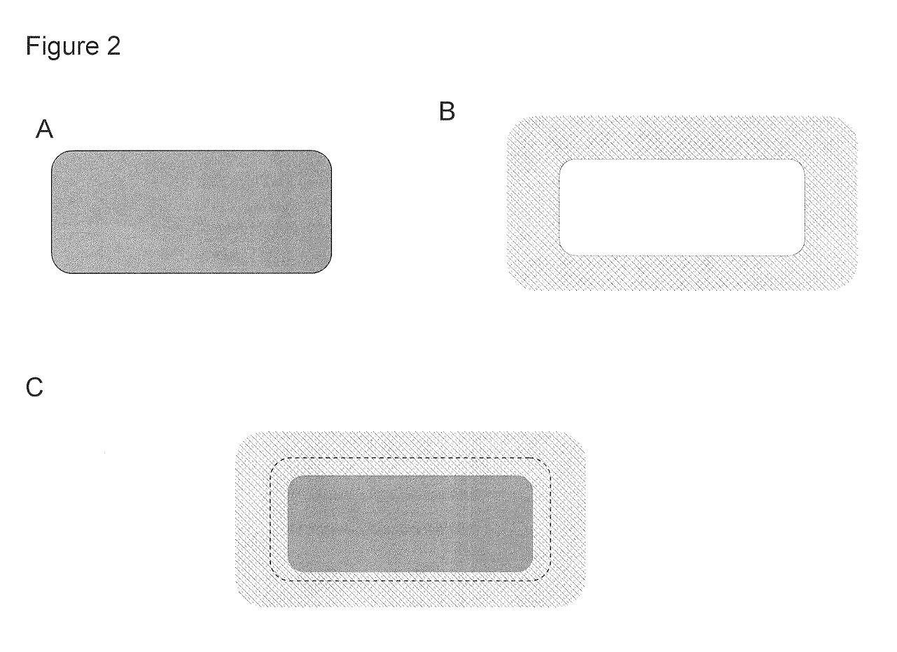

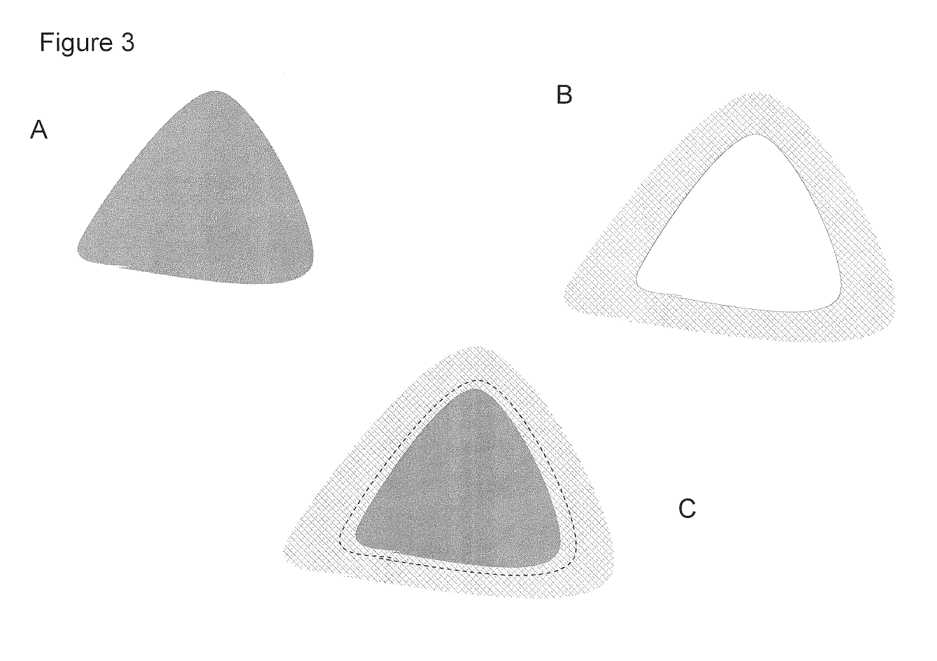

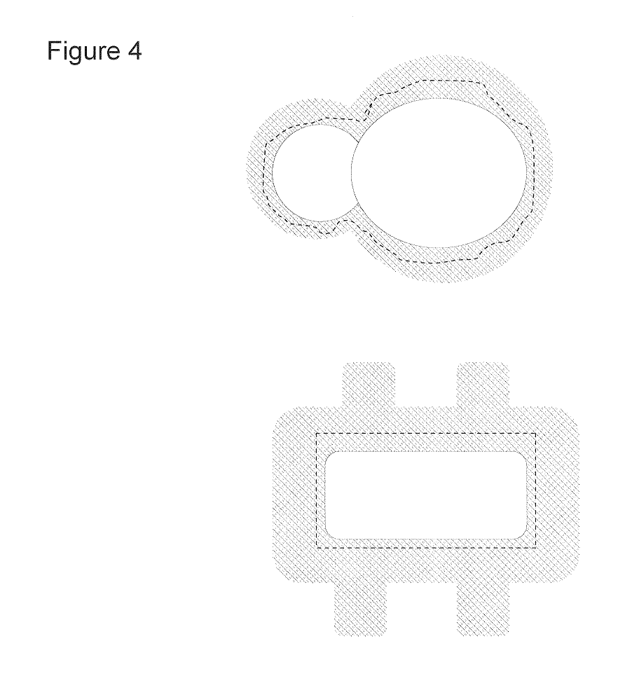

The size and shape of a wound dressing according to an embodiment of the present invention can vary depending on the wound the dressing is to be applied. Additional wound dressings of various sizes and shapes that can be used in embodiments of the present invention are illustrated in FIGS. 2-4. The wound dressing can be, for example, rectangular, triangular, circular, or donut-shaped. As readily appreciated by those skilled in the art in view of the present disclosure, other shapes and sizes of the wound dressings are also possible.

In one embodiment of the present invention, one or more corners of the allograft are rounded or flattened to prevent the corners from catching the wound during application. In view of the present disclosure, any method known to those skilled in the art can be used to make the corners of the allograft round or flattened.

In one embodiment of the present invention, the allograft, and optionally also the base sheet, in the wound dressing can carry one or more therapeutic agents, such as growth enhancing agents, stem or progenitor cells, morphogenic proteins, small molecule compounds, pharmaceutical agents, anti-microbial agents, agents that prevent scarring, adhesions and tethering at or near the wound site, analgesics, etc., to further improve wound healing. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

The base sheet is generally conformable to the surface where it applies. The base sheet can be made of any suitable materials in view of the present disclosure, such as those surgical fabric meshes approved by the Food and Drug Administration (FDA). Examples of materials suitable for the base sheet include, but are not limited to, any medical grade polymer or monomer, polyvinyl alcohol (PVA), polytetrafluroethylene (PTFE), expanded polytetrafluroethylene (ePTFE), porous polyethylene, porous polypropylenes, liquid polyurethanes for hollow-fiber implants, polyurethanes for dip-molding, biostable polyurethanes, thermoplastic polyurethanes, etc.

Various adhesives, preferably pressure sensitive adhesives, can be used to form an adhesive layer on the base sheet to make it adhesive to the skin, the allograft, and/or the support layer. The adhesive used on the first major surface of the base sheet, which is to be in contact with the skin, is skin compatible and hypoallergenic, such as the surgical adhesives approved by the FDA. Examples of the adhesives include, but are not limited to, cyanoacrylates, fibrin sealants, gelatin and thrombin products, polyethylene glycol polymers, albumin and glutaraldehyde products, or other suitable adhesives known in the art in view of the present disclosure. Preferably, the adhesives are specific to the tissue where the wound dressing is to be applied.

Optionally the wound dressing also includes a support layer mounted on a second major surface of the base sheet opposite to the first major surface where the allograft adheres. Preferably, the support layer is resistant to the passage of microbes therethrough. It can be a translucent or transparent polymeric elastic film so that the condition of the allograft applied to the wound site can be viewed through without removing the support layer. Preferably, the support layer is permeable to moisture. The material used to form the support layer is generally substantially more rigid than that used to form the base layer, e.g., to prevent the support from improperly wrinkling during application to a wound site. The support layer materials can include, but are not limited to, polyethylene/vinyl acetate copolymer-coated papers, polyurethane or polyester films and other medical grade polymer or monomer films. The support layer can be affixed to the base sheet with an adhesion coating or by another means, such a bandage. The support layer can also provide additional protection to the wound.

According to embodiments of the present invention, the allograft can be a single layer of amnion or chorion, more than one layer of amnion or chorion, or a combination of one or more layers of amnion and one or more layers of chorion. When the allograft is a combination of one or more layers of amnion and one or more layers of chorion, the layers can be arranged in any order. The multiple layers in the allograft can also be subject to a cross-linking treatment to make the layers closely adhere to each other in an integrated form. In allograft comprising a combination of multiple layers of amnion and multiple layers of chorion, the layers can be arranged in any order, and can be in any combination.

Preferably the allograft of the wound dressing has thickness of 0.02 mm to 0.10 mm.

In another general aspect, embodiments of the present invention relate to a method of preparing a wound dressing. The method comprises: drying an allograft comprising at least one layer of human amnion and chorion tissues over a frame of a shape appropriate for covering a wound; obtaining a base sheet having an adhesive on a first major surface of the base sheet and a central opening extending therethrough, the opening having a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound; sterilizing the allograft and the base sheet; and assembling the allograft together with the base sheet.

Optionally, the method further comprises obtaining a support layer and assembling the support layer together with the allograft and the base sheet.

The frame can be a resorbable frame, e.g., polymer mesh frame, or a disposable or stainless steel frame. Preferably, the frame is rigid or semi rigid. The frame can be any of the shapes suitable for wound dressings, e.g., round, rectangle, triangle, etc.

In an embodiment of the present invention, when a disposable frame is used, the dried tissue retains the shape of the frame when removed from the frame. The allograft could be packaged and sterilized with or without the disposable frame. The disposable frame can be removed and discarded prior to the use as part of the wound dressing. The disposable frame can be longer than the tissue for ease of handling and removal.

In another embodiment of the present invention, an implantable and resorbable frame is used. Such frame can be a mesh or a solid frame with several holes throughout, and can be used together with the allograft in the wound dressing.

The allograft, such as that comprising one or more layers of human amnion and/or chorion tissues, is bonded to the frame by various methods in view of the present disclosure, such as, drying the tissue on the frame, using a resorbable adhesive, keeping the tissue wet and laying it on the frame, or freezing the tissue on the frame.

Preferably, one or more corners of the allograft are rounded or flattened using methods known in the art in view of the present disclosure.

The base sheet and support layer can be obtained, and assembled together with the allograft, by methods known in the art in view of the present disclosure.

The allograft, base sheet and support layer can be assembled together in one package, such as a laminated peel pouch, paper peel pouch, sealed with a laminated cover. They can each be individually wrapped before they are assembled together. They can be sterilized by methods known to those in the art, such as gamma irradiation.

Another aspect of the present invention relates to the use of a wound dressing according to an embodiment of the present invention to improve wound healing, by applying the wound dressing to a wound.

The application of a wound dressing of the present invention to a wound can prevent scarring, reduce inflammation, inhibit microbial infection and improve healing. The allograft also has the ability to enhance wound healing, reduce scar formation, inflammation and risk of infection.

Prior to the application of the wound dressing, the wound is cleansed with a sterile solution of normal saline. The wound can also be cleansed with an amniotic fluid. Preferably, the amniotic fluid is processed so that it has a relatively high viscosity for ease of application and for remaining in the desired area after the application. Methods known to those skilled in the art can be used to prepare amniotic fluid with a relatively high viscosity in view of the present disclosure. A wound dressing according to an embodiment of the present invention is then applied to the wound.

In one embodiment of the present invention, the allograft of the appropriate shape and size is first applied to the wound site; the base sheet is then applied to secure the allograft over the wound site. When needed, the support layer is applied to protect the allograft. The support layer is mounted to the base sheet by an adhesive or secured with tape or a suitable compression bandage, as appropriate.

In another embodiment of the present invention, the allograft further comprising an implantable and resorbable frame is first applied to the wound site followed by application of the base sheet to secure the allograft over the wound site.

In another yet embodiment of the present invention, the allograft, the base sheet, and optionally the support layer, are first assembled into a wound dressing, and the wound dressing is then applied to the wound site.

According to embodiments of the present invention, an allograft comprising a combination of multiple layers of human amnion and chorion tissues can be applied to a wound with either the amnion or chorion tissue directly contacting the raw dermis of the wound. Amnion tissue has two surfaces: (1) an outer surface in contact with chorion tissue; and (2) an inner surface in contact with amniotic fluid. Likewise, chorion tissue also has two surfaces: (1) an outer surface that is contact with maternal cells; and (2) and inner surface that is in contact with amnion tissue. According to embodiments of present the present invention, either surface (i.e. inner or outer) of either tissue of the allograft (i.e. amnion or chorion) can directly contact the raw dermis of a wound when a wound dressing of the present invention is applied to the wound.

The wound dressings according to embodiments of the present invention can be applied to any wound site in view of the present disclosure. A size of wound dressing is chosen that will overlap the edges of the wound, preferably by 2-3 cm. The frequency with which the wound dressing should be changed depends upon the nature of the wound and the amount of exudate produced. On a clean non-infected wound, it may be left in position for up to five or six days, but more frequent changes will be required on infected or very heavily exuding wounds.

The present invention overcomes shortcomings of the prior art by making human allograft membranes usable as wound dressings.

There are several attributes which make an allograft having at least one of amnion and chorion tissues a preferred material for use in wound healing. Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J Ophthalmol. 85(4):444-9) and produces natural inhibitors of matrix metalloproteases (MMPs) expressed by infiltrating polymorphonuclear cells and macrophages. Hao et al., 2000, Cornea, 19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it a potential allograft candidate to be used in wound healing.

Human allograft amnion and chorion have the ability to prevent scarring, reduce inflammation, inhibit microbial infection and improve healing. The allograft has the ability to enhance wound healing, reduces scar formation, inflammation and risk of infection.

Amnion, chorion and amniotic fluid used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile bag, which is placed in an ice bucket, and is delivered to another location. The placenta is rinsed, e.g., with sterile saline, to remove excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The prepared amnion and/or chorion can be sized into various sizes and shapes anticipated to be useful in a wound dressing according to embodiments of the present invention.

The present invention encompasses a kit comprising a plurality of wound dressings according to an embodiment of the present invention, wherein at least two of the plurality of wound dressings are different in at least one of size and shape. The wound dressing can further comprise one or more therapeutically active agents, such as anti-microbial agents, growth enhancing agents, anti-inflammatory agents, analgesics, etc.

According to an embodiment of the present application, the kit further comprises an amniotic fluid and instructions on how to use the amniotic fluid in the wound healing. Preferably, the amniotic fluid is processed amniotic fluid having a relatively high viscosity.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A wound dressing comprising:
(1) an allograft having at least one layer of human amnion and chorion tissues and a size and shape appropriate for placing inside boundaries of a wound; and
(2) a base sheet having an adhesive on a first major surface of the base sheet and a central opening extending therethrough, wherein the opening has a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound; and
(3) optionally a support layer mounted on a second major surface of the base sheet opposite to the first major surface.

2. The wound dressing of claim 1, wherein the support layer is resistant to the passage of microbes therethrough.

3. The wound dressing of claim 1, further comprising one or more therapeutic agents to further improve the wound healing.

4. The wound dressing of claim 3, wherein the one or more therapeutic agents are selected from the group consisting of growth enhancing agents, stem or progenitor cells, morphogenic proteins, small molecule compounds, pharmaceutical agents, anti-microbial agents, agents that prevent scarring, adhesions and tethering at or near the wound site, and analgesics.

5. The wound dressing of claim 1, wherein one or more corners of the allograft are rounded or flattened.

6. A method of preparing a wound dressing, the method comprising:
drying an allograft comprising at least one layer of human amnion and chorion tissues over a frame having a shape appropriate for placing inside boundaries of a wound;
obtaining a base sheet having an adhesive on a first major surface of the base sheet and a central opening extending therethrough, the opening having a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound;
sterilizing the allograft and the base sheet; and
assembling the allograft and the base sheet in a package.

7. The method of claim 6, wherein the human amnion and chorion tissues are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining amnion and chorion tissues from the placenta.

8. The method of claim 7, further comprising:
obtaining a support layer; and
assembling the support layer together with the allograft and the base sheet.

9. The method of claim 6 further comprising rounding or flattening one or more corners of the allograft.

10. A method of improving wound healing, comprising applying to a wound a wound dressing comprising:
(1) an allograft having at least one layer of human amnion and chorion tissues and a size and shape appropriate for placing within boundaries; and
(2) a base sheet having an adhesive on a first major surface of the base sheet and a central opening extending therethrough, wherein the opening has a dimension smaller than the dimension of the allograft such that when the wound dressing is applied to the wound, the base sheet adheres to the skin surrounding the wound and the periphery of the allograft to thereby secure the allograft within the boundaries directly on raw dermis of the wound; and
(3) optionally a support layer mounted on a second major surface of the base sheet opposite to the first major surface.

11. The method of claim 10, wherein the allograft and the base sheet, and optionally the support layer, are applied to the wound separately.

12. The method of claim 10, wherein the allograft and the base sheet, and optionally the support layer, are first assembled together, then applied to the wound.

13. The method of claim 10, wherein the wound dressing further comprises one or more therapeutic agents to further improve the wound healing.

14. The method of claim 10, wherein one or more corners of the allograft are rounded or flattened.

15. The method of claim 10, further comprising administering to the wound an amniotic fluid.

16. The method of claim 15, wherein the amniotic fluid is processed amniotic fluid having a relatively high viscosity.

17. A kit comprising a plurality of wound dressings according to claim 1 and instructions on how to use the wound dressings in wound healing, wherein at least two of the wound dressings are different in at least one of size and shape.

18. The kit of claim 17 further comprising an amniotic fluid and instructions on how to use the amniotic fluid in wound healing.

19. The kit of claim 18, wherein the amniotic fluid is processed amniotic fluid having a relatively high viscosity.
